(19) United States
(12) Patent Application Publication
(10) Pub. No.: US 2014/0317884 A1
(43) Pub. Date: Oct. 30, 2014

(54) GRIPPING SYSTEM FOR MEDICAL DEVICES TO IMPROVE HAPTICS AND PREVENT HAND FATIGUE

(71) Applicant: TODD J. COHEN, Port Washington, NY (US)

(72) Inventor: TODD J. COHEN, Port Washington, NY (US)

(21) Appl. No.: 14/215,362

(22) Filed: Mar. 17, 2014

Related U.S. Application Data
(60) Provisional application No. 61/798,162, filed on Mar. 15, 2013.

(51) Int. Cl. A61B 17/00; B25G 1/02

(52) U.S. Cl. CPC A61B 17/00; B25G 1/02; A61B 2017/00424
USPC 16/421

(57) ABSTRACT

A resilient gripping member or medical device can be applied to a handle or other gripping area of a firmer medical device to provide improved cushioning and comfort and uniformity of feel to an operator's hand during manual manipulation. The resilient gripping member or medical device may be tubular and comprise at least one control, button, switch, or sensor that communicates with another component not directly located on the firmer medical device.

### CROSS-REFERENCE TO RELATED APPLICATION

This application is based upon and claims the benefit of the filing date of co-pending, commonly assigned U.S. Provisional Patent Application Ser. No. 61/798,771, filed Mar. 15, 2013, incorporated herein by reference in its entirety.

### FIELD OF THE INVENTION

This invention is directed to a gripping system for medical devices. More particularly, this invention is directed to a medical system that improves haptics and prevents or minimizes hand fatigue.

### BACKGROUND OF THE INVENTION

Medical devices themselves are often made of molded or shaped stainless steel or molded plastics. The shapes of such devices are sometimes arbitrary. Without sterile gloves, these devices can be slippery and difficult to hold. Even with sterile gloves, holding them often is uncomfortable and may contribute to a confusing feeling, or an uncomfortable sense of touch if not hand fatigue.

The sense of touch as it relates to the operation of medical devices and surgery is known as haptics. The loss of this sense has been a problem for the remote performance of surgery or procedures, specifically in the field of robotics.

In the operating room, surgery is performed with a myriad of surgical instruments and tools, including needle drivers, forceps, mosquito clamps, weitlaner retractors, pick-ups, etc. Many standard surgical instruments may be seen at the following link: http://www.surgical-instrument-pictures.com/instrument-index.html.

Standard surgical instruments are often comprised of stainless steel. Operators and their assistants will often retract an open wound or firmly hold a stainless steel medical device, which can lead to finger, hand, wrist, or arm fatigue. In addition, some repetitive motions, such as suturing, could themselves also lead to repetitive stress syndrome.

In the interventional cardiology field of electrophysiology, cardiac ablation catheters of each manufacturer, and even within each manufacturer, often have handles of different shapes (although their sizes may be similar). Moreover, the catheters are constructed such that they are gripped with either a firm plastic (in one instance, St. Jude Medical) or additional firm rubber (i.e., composite composition) on the side where it is gripped. The different shapes may be loosely cylindrical but differ significantly.

Biosense Webster makes a catheter called the EZ Steer™, which is somewhat polygonal on the side of the deflection knob and mildly curved on the sides opposite the deflection knob. In contrast, the Boston Scientific Blazer II XP Register™ catheter is flat on the side of the deflection knob. The St. Jude Medical Safire™ catheter has a more unusual reverse conical design, but it has curved rubber on the sides and plastic on the top where the name appears. Other catheter handles have a cylindrical plastic shaft with a pull knob. The diameters of each of these handles range from about 0.7 inch to about 2 inches.

During ablation cases, the ablation catheters may be held and rotated countless times, and each of these catheters has no uniformity of feel, and very little give. In addition, there are features that may need to be applied to these catheters such as switches, functionality, and accessories, which if added would confuse, tangle, and affect manipulation and gripping rotation and possibly dislodge an applied accessory to or on the ablation catheter itself.

Some non-medical areas are different. For example, in the field of fishing, manufacturers have created a more uniform feel to a fishing rod rather than making the handle purely plastic or only a continuation of a rod's shaft. The addition of cork to the handle of a fishing rod provides some circumferential give, but not as much as the application of a rubber polymer or copolymer or foam or foam composite to the rod handle. This application has provided some give in the rod handle and allows an angler to deal with long holds and increased pressure from the grip during the “catch and release” without suffering much from hand pain, fatigue, discomfort, and repetitive stress.

There is a need for many medical devices (such as endoscope probes, and lasers), surgical tools (such as hand-held retractors), and even catheter ablation handles, to have the ability to have either a built-in or a self-applied rubber polymer cushion of some form to improve the feel and prevent pain and fatigue from repetitive motion.

### SUMMARY OF THE INVENTION

In accordance with the invention, a gripping member is designed to slide over or be wrapped around the handle of a medical device, to provide comfort and feel. Preferably the gripping member, or gripper, will comprise a sterilizable, disposable material.

In one embodiment of the invention, on the handle of a sterile medical device, such as an ablation catheter, where the device is gripped, there is a sterile, disposable gripping member, or gripper, that slides over the handle, or alternatively wraps around the handle, to provide a uniform circumferential giving feel to the handle. In the case of a catheter, the gripping member can slide over either the entire catheter handle or only a portion of it, such as the back. The gripping member comprises rubber, foam, or some other resilient or givable material. Additionally, the material must be sterilizable for operation in a sterile medical field. More typically, the gripper would be a disposable medical device.

Approximately four inches of the ablation catheter's handle would need to be covered to provide a suitable grip for a user. In one embodiment, a stretchable rubber cylindrical sterile tube would be pulled onto the rear or proximal end of a catheter and stretched over the rear connector onto the handle such that it covers the entire gripping surface. This cylindrical gripper device could be configured to fit on a variety of similarly sized ablation catheter handles (such as the Biosense Webster EZ Steer™ and the Boston Scientific Blazer II XP Register™ catheter). In other instances, the gripper would need to be separately configured to the design of the catheter due to a significant size and shape handle configuration (such as the St. Jude Medical Safire™ catheter),

In another embodiment of the invention, if a switch, button, or wired accessory is attached to a device handle, the gripping member could obscure, hide, and prevent any disruption of feel or entanglement from any hidden wire beneath the gripping member.

In another embodiment of the invention, the gripping member could be more rectangular or non-cylindrical, In such a case the gripping member would be pulled over and wrapped around the handle and then attached with some form of adhesive or adhesive tape, or even VELCRO® hooks or another known fastening means. Alternatively the gripping member could wrap around the handle in a spiral fashion, similar to a tennis racket handle, and be secured with sterile tape at its proximal and distal ends or via self adhesive.

Applying a gripping member to a catheter handle would cause a slight cushioned feel circumferentially about the catheter handle, which would be relatively uniform regardless of the catheter handle design. It would provide a cushioned feel and conceal any wires or switches or buttons, which would need to be applied to the front of the catheter.

In another embodiment of the invention, a cushioned gripping member itself could have incorporated into the rubber polymer or foam as part of or as a complete accessory such as a sensor, button, wire to a button or switch, or some other controller or controlling mechanism. This would permit maximal manual operation without entanglement from hardwiring of an attached accessory to the catheter. It would also provide additional functionality to the medical device. Optionally the controller could be wireless.

In another embodiment of the invention, a cushioned flexible gripping member may include an accessory switch to a ablation catheter switching mechanism. A circumferential end of the gripping member could slip over the front or rear end of the ablation catheter handle such that a self-contained switching mechanism would be located near the thumb position while holding the ablation catheter handle. The same gripping member could stretch back and fold over and around the handle and attach to a separate switching mechanism controller. Any wires, switches and sensors could be concealed within a rubber or foam construction of the gripping member.

With respect to surgical devices such as hand-held metal retractors, the application of a sterile rubber polymer or foam to the underside or hand gripping side or hand held application component would help minimize hand fatigue during prolonged surgical retractions or holds. An embodiment of the invention useful here could simply be a sterile disposable stretchable rubber tube or wrap around instrument handle which could slip over the handle of the retractor and help cushion the experience. Alternatively, a adhesive sterile polymer could attach to areas of stainless steel medical instruments and retractors and be applied to them once on the surgical sterile field. Their use could minimize hand fatigue and discomfort during the procedure. Smaller rubber tubes could slide over forceps' ends, retractors, and the like and provide more hand comfort, control, and uniformity of feel.

In another embodiment of the invention, a resilient gripping member or medical device can be applied to a handle or other gripping area of a firmer medical device to provide improved cushioning and comfort and uniformity of feel to an operator's hand during manual manipulation.

In another embodiment of the invention, a resilient gripping member or medical device comprises a rubber polymer or copolymer or foam or foam composite and can stretch or wrap around and secure itself over the handle or other gripping area to provide comfort and improve tactile feel to the operator.

In another embodiment of the invention, a resilient gripping member or medical device is sterile or sterilizable.

In another embodiment of the invention, a resilient gripping member or medical device has an adhesive end for attaching to the handle or other gripping area to provide a cushion to the handle or other gripping area.

In another embodiment of the invention, a resilient gripping member or medical device is built into a firmer medical device for the purpose of improved comfort and feel during manipulation.

In another embodiment of the invention, a resilient gripping member or medical device comprises a built-in part or component or complete accessory component for attachment to a firmer medical device.

In another embodiment of the invention, the firmer medical device is a sterile surgical instrument such as a retractor, a clamp, or forceps.

In another embodiment of the invention, the firmer medical device is a nonsterile medical instrument such as an endoscope, cystoscope, protoscope, or bronchoscope.

In another embodiment of the invention, the firmer medical device is a sterile medical catheter with a handle or firm gripping surface such as a cardiac ablation catheter

In another embodiment of the invention, a resilient gripping member or medical device is contoured to slide onto or attach to a specific portion of a firmer medical device to enhance the manual comfort during operation of the firmer medical device.

In another embodiment of the invention, a resilient gripping member or medical device is tubular in shape so that it may slide onto a hand-held component of a firmer medical device to enhance comfort and prevent hand fatigue.

In another embodiment of the invention, a resilient gripping member or medical device is comprised of an outer cushioned surface and an attachable or securing component to secure itself to the medical device.

In another embodiment of the invention, in a resilient gripping member or medical device the attachable or securing component is adhesive.

In another embodiment of the invention, in a resilient gripping member or medical device the attachable or securing component is a VELCRO substrate.

In another embodiment of the invention, in a resilient gripping member or medical device the contour, shape, and size is specific to the specific contour, shape, and size of the firmer hand-held medical device.

In another embodiment of the invention, a resilient gripping member or medical device is two-dimensional in design and wraps around a medical device and secures one end to another via adhesive, VELCRO, hook, fastener, or some other attachment to provide a cushioned feel over the handheld component of a firm medical device.

In another embodiment of the invention, a resilient gripping member or medical device contains control, switches, and/or sensors, to add functionality to a firmer medical device that communicates with another component such as a switching mechanism or controller not directly located on the firmer medical device.

In another embodiment of the invention, a resilient gripping member or medical device comprises at least one control, button, switch, or sensor to add functionality to a firmer medical device that communicates with another component not directly located on the firmer medical device.

In another embodiment of a resilient gripping member or medical device of the invention, the other component is a switching mechanism or controller.

In another embodiment of a resilient gripping member or medical device of the invention, a gripping means suitable for the handle of an ablation catheter comprises an actuator that communicates with ablation therapy.

In another embodiment of a resilient gripping member or medical device of the invention, the actuator immediately disconnects ablation therapy.

Before at least one embodiment of the invention is explained in detail, it is to be understood that the invention is not necessarily limited in its application to the details of construction and the arrangement of the components and/or methods set forth in the following description and/or illustrated in the drawings and/or the examples. The invention is capable of other embodiments or of being practiced or carried out in various ways.

### BRIEF DESCRIPTION OF THE DRAWINGS

FIGS. 1A to 1D are each a schematic representation of a gripping member according to the invention;

FIG. 2 is a schematic representation of a gripping member of the invention applied to a retractor;

FIG. 3 is a schematic representation of a gripping member of the invention applied to forceps;

FIG. 4 is a schematic representation of a quick disconnect release unit for use with an ablation catheter where there is a remote activation button;

FIG. 5 is a schematic representation of a detail of FIG. 4; and

FIG. 6 is a schematic representation of a safety release unit similar to that shown in FIG. 4 where the remote activation button has been incorporated into a gripping member attached to an ablation catheter handle.

### DETAILED DESCRIPTION OF THE INVENTION

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced. It should be understood that the invention is not necessarily limited in its application to the details of construction and the arrangement of the components and/or methods set forth in the following description and/or illustrated in the drawings. The invention is capable of other embodiments or of being practiced or carried out in various ways.

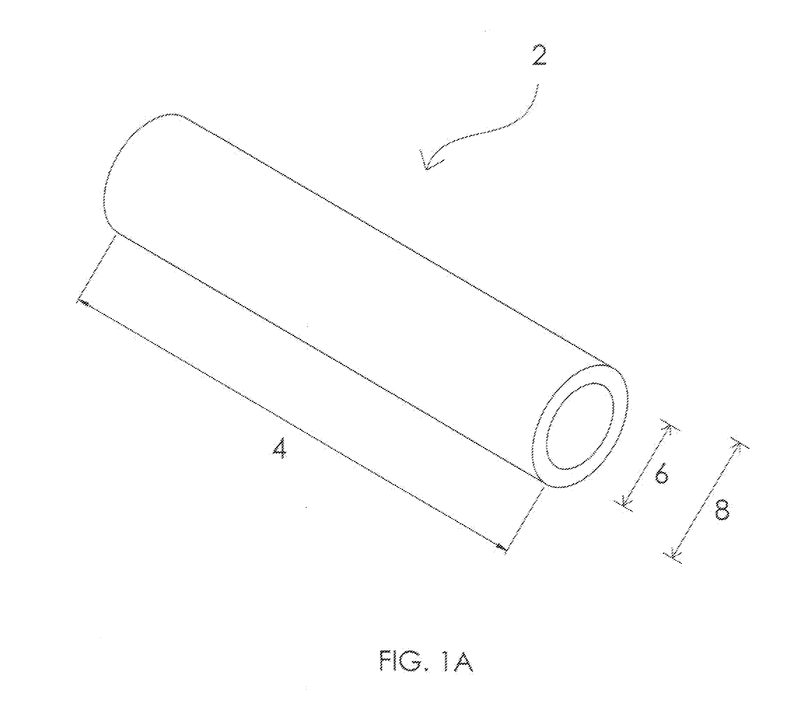

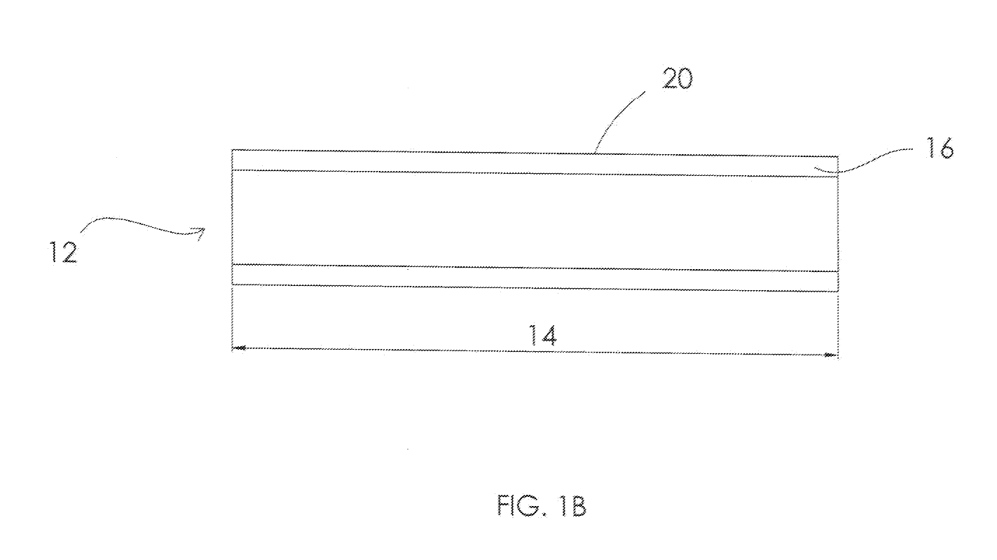

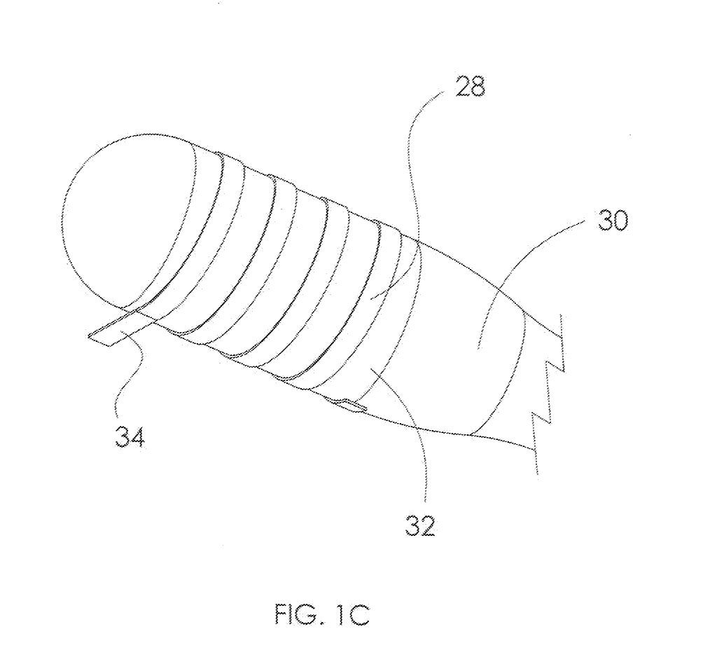

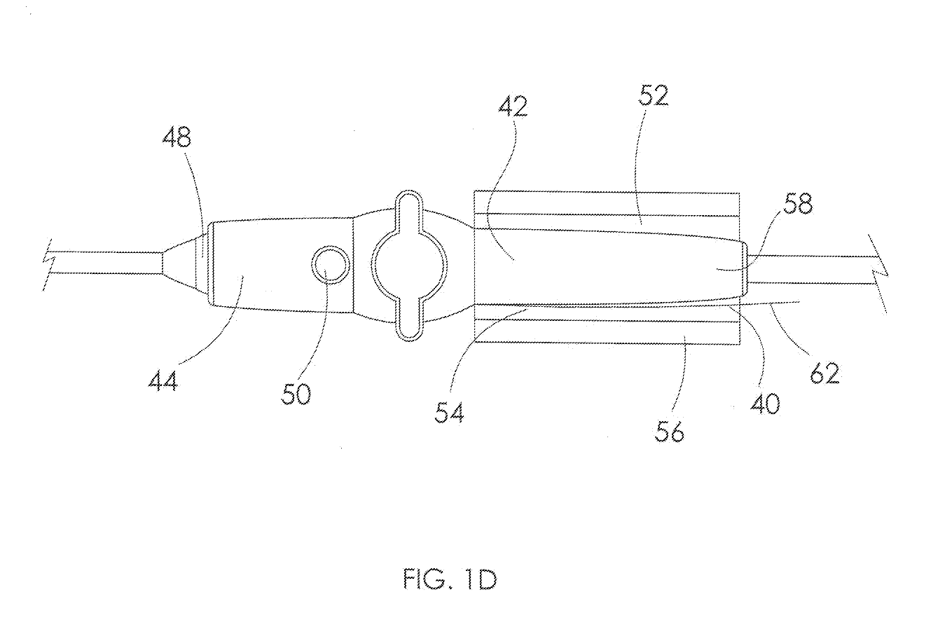

FIG. 1A is a schematic representation of a gripping member 2 suitable for and sized to fit tightly over the handle of an ablation catheter (not shown), such as the Boston Scientific BLAZER or the Biosense Webster EZ Steer. The longitudinal length 4 of gripping member 2 is about 4 inches, and the internal diameter 6 is about 0.5 inches. The external diameter 8 is about 0.7 inches.

The embodiment of the invention represented in FIG. 1B is a gripping member 12 that is about four inches in length 14 and has adhesive 16 on at least one lateral edge 20. Gripping member 12 is designed to partly or completely wrap around an ablation catheter handle (not shown).

FIG. 1C is a schematic of a gripping member 28 that spirals around a catheter handle 30, similar to the way in which a grip is applied to a tennis racket handle. The ends 32, 34 of gripping member 28 are secured to handle 30 by suitable means, such as adhesive, adhesive tape, or a rubber sleeve (not shown).

In FIG. 1D, a gripping member 40 is contoured to fit around an ablation catheter handle 42. The distal portion 44 of gripping member 40 is formed to surround the distal portion 48 of catheter handle 42 or is in one section or in two sections that are fastened or glued together. Distal portion 44 can fit around a button 50 on handle 42, which button can be a kill switch. The proximal portion 52 of gripping member 40 has two lateral portions 54 that adhere with adhesive or other fastening means 56 either to each other or to the proximal portion 58 of handle 42. Optionally gripping member 42 may have a built-in wire 62 that, for example, connects to button 50.

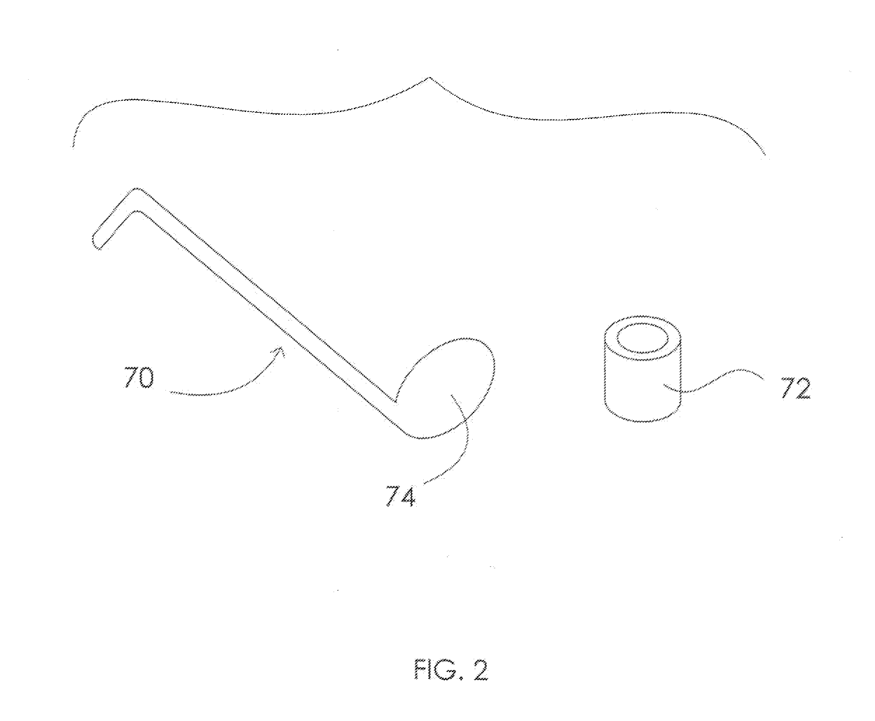

FIG. 2 is a schematic representation of a stainless steel surgical retractor 70 and a gripping member 72. Gripping member 72 is of appropriate dimensions so that it slides over a grip 74 of retractor 70 and snuggly retains its position while the retractor is in use.

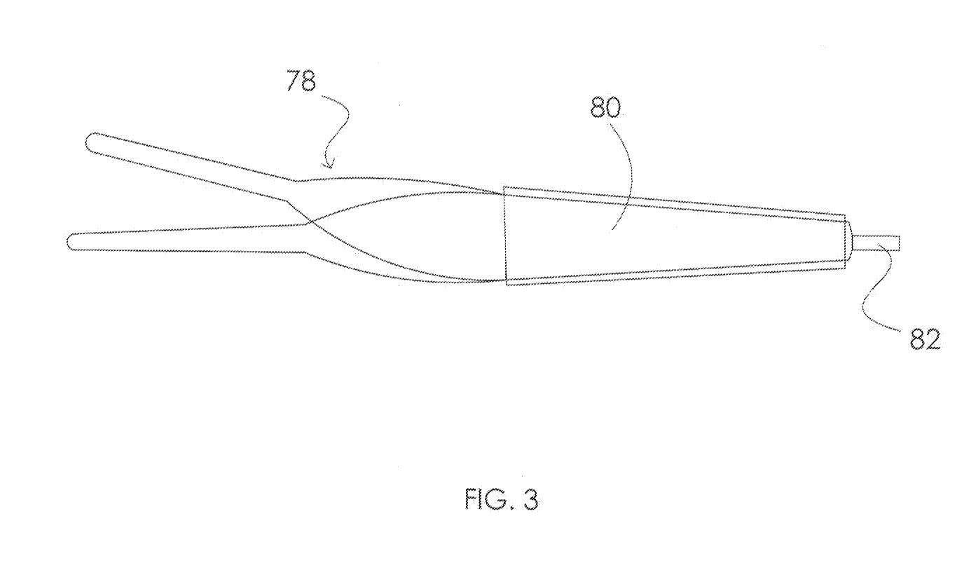

FIG. 3 is a schematic representation of typical forceps 78 used in surgical or clinical procedures. A gripping member 80 slides over and snuggly fits on the proximal handle section 82 of forceps 78.

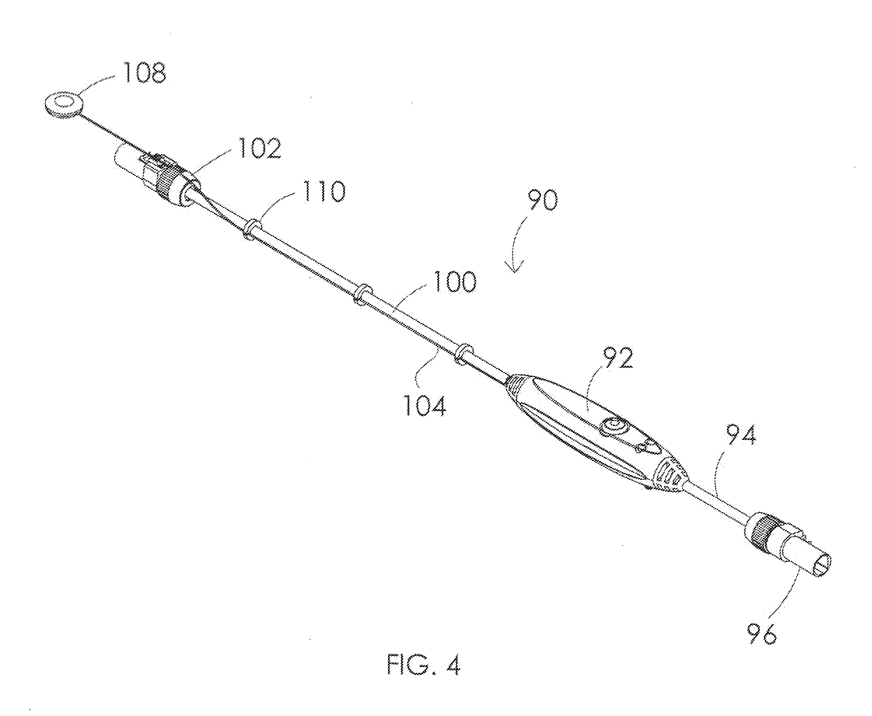

In FIG. 4, a quick disconnect/release system 90 comprises an electronics module 92 with a proximal cable 94 having a connector 96 and a distal cable 100 having a connector 102. A wire 104 extends from module 92 to an activation button 108. Wire 104 is secured to distal cable 100 with adhesive or two or more ties, clips, or comparable securing devices 110.

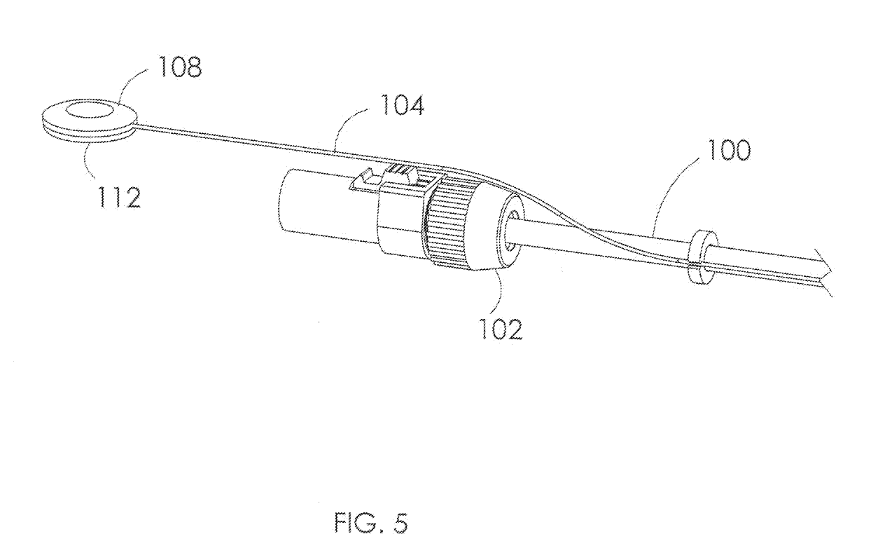

Activation button 108 is configured to attach to a catheter handle, such as is shown in FIG. 5. For example, as shown more clearly in FIG. 5, the bottom 112 of activation button 108 may have an adhesive surface covered by a peel-away plastic strip (not shown).

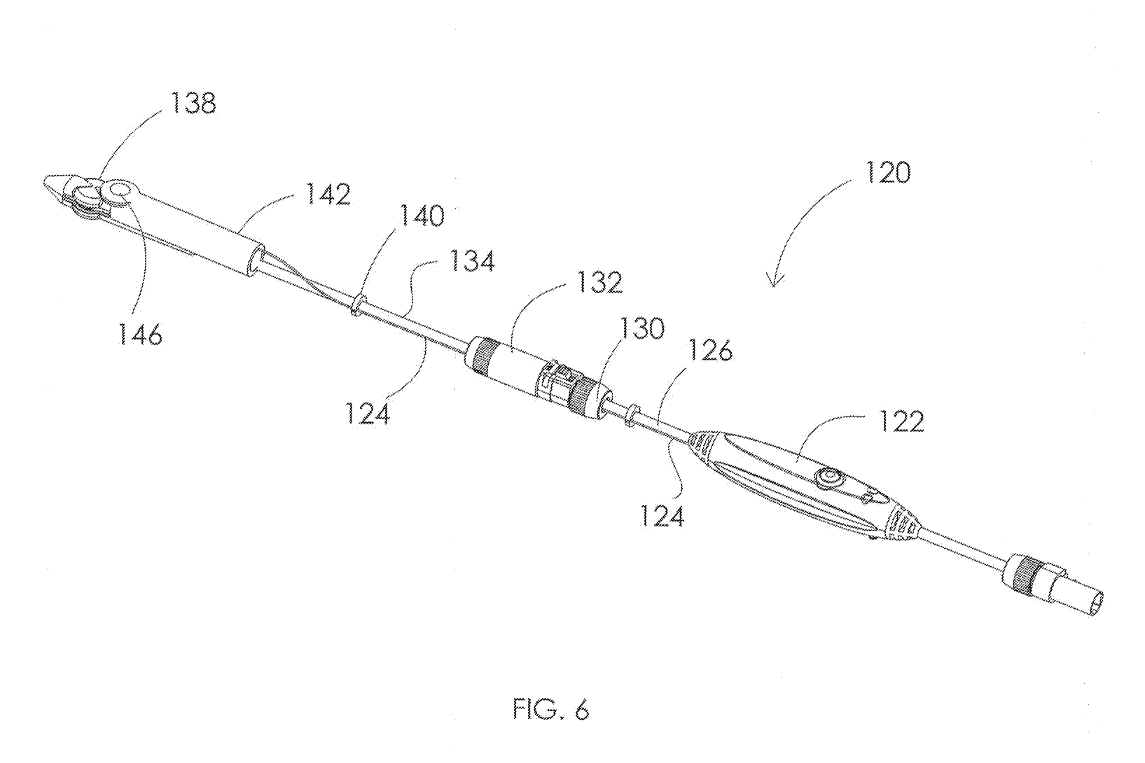

A gripping member according to the invention may coordinate with an activation button, as shown in FIG. 1, or an activation button may be incorporated into a gripping member. As shown in FIG. 6, a quick disconnect/release system 120 comprises an electronics module 122 with a distally extending wire 124 that extends along cable 126 and passes through connector 130 into a connector 132 on a “pigtail” cable 134 that in turn is connected to an ablation catheter handle 138. Adhesive or two or more ties, clips, or comparable securing devices 140 secure wire 124 to cables 126 and 134.

Wire 124 extends into gripping member 142, which has been snuggly fitted around catheter handle 138. Gripping member 142 has an activation button 146 that is in electrical communication with wire 124.

When an ablation catheter handle does not have a pigtail cable but instead has a connector 132 at the proximal end of the ablation catheter handle (not shown), the distal portion of wire 124 will be shorter so that there is no portion of wire 124 that is not secured to a gripping member or a cable. It is important that the wire be neatly and appropriately constrained to keep it from binding, tangling, or otherwise interfering with movement of the catheter or a cable.

Useful materials for the gripping member include any number of sterilizable resilient polymeric foams and rubbers. One material that seems to be particularly useful is ethyl vinyl acetate (EVA), although many others, such as NEOPRENE, are useful as well. Examples of other useful materials include thermoplastic elastomers (TPEs), such as thermoplastic vulcanates (rubber polyolefin blends), polyetheramides, polyesters, styrene-ethylene-butylene-styrene (SEBS) block copolymers, styrene-butadiene-styrene (SBS) block copolymers, partially or fully hydrogenated styrenebutadiene-styrene block copolymers, styrene-isoprene-styrene (SIS) block copolymers, partially or fully hydrogenated styrene-isoprene-styrene block copolymers, polyurethanes, polyolefin elastomers, polyolefin plastomers, styrenic based polyolefin elastomers, compatible mixtures thereof, and similar thermoplastic elastomers. SEBS, SBS and SIS block copolymers are commercially available from Shell under the tradename KRATON rubber. Other suitable thermoplastic elastomers include, e.g., KRATON rubber-based block copolymers such as DYNAFLEX G2701 and DYNAFLEX G2755 polymers, commercially available from GLS Corp., Cary, Ill. Other suitable resilient materials include the PEBAX® family of polymers (available from Elf AtoChem, Philadelphia, Pa. which can be used pure or as blends), resilient urethanes, silicones, rubbers, and foams. Suitable foams include polyurethane foams, e.g., those prepared from compositions having two components: a foamable, curable polyurethane prepolymer, and an aqueous phase containing a latex and a surfactant.

The gripping member for a cardiac ablation catheter should be about four inches in length and have an effective diameter of from about 0.4 inches to about 2 inches. There may be instruments where the handles are smaller or, most likely, larger, so that a gripping member may be quite different in size and configuration which is certainly within the scope of the invention.

The embodiments shown in the drawings are representative of ways in which a control button, activation button, sensor, or other device or control could be incorporated into a gripping member for an ablation catheter handle or other portion of a medical device. For example, sensors incorporated into a gripping member could sense ambient temperature, an operator's body temperature, humidity, heart rate, or other parameters of the environment or the operator.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed:

1. A resilient medical device which can be applied to a handle or other gripping area of a firmer medical device to provide improved cushioning and comfort and uniformity of feel to an operator's hand during manual manipulation.

2. The resilient medical device of claim 1 which comprises a rubber polymer or copolymer or foam or foam composite and can stretch or wrap around and secure itself over the handle or other gripping area to provide comfort and improve tactile feel to the operator.

3. The resilient medical device of claim 1 which is sterile or sterilizable.

4. The resilient medical device of claim 1 which has an adhesive end for attaching to the handle or other gripping area to provide a cushion to the handle or other gripping area.

5. The resilient medical device of claim 1 which is built into a firmer medical device for the purpose of improved comfort and feel during manipulation.

6. The resilient medical device of claim 1 which comprises a built-in part or component or complete accessory component for attachment to a firmer medical device.

7. The resilient medical device of claim 1, wherein the firmer medical device is a sterile surgical instrument such as a retractor, a clamp, or forceps.

8. The resilient medical device of claim 1, wherein the firmer medical device is a nonsterile medical instrument such as an endoscope, cystoscope, protoscope, or bronchoscope.

9. The resilient medical device of claim 1, wherein the firmer medical device is a sterile medical catheter with a handle or firm gripping surface such as a cardiac ablation catheter

10. The resilient medical device of claim 1 which is contoured to slide onto or attach to a specific portion of a firmer medical device to enhance the manual comfort during operation of the firmer medical device.

11. The resilient medical device of claim 1 which is tubular in shape so that it may slide onto a hand-held component of a firmer medical device to enhance comfort and prevent hand fatigue.

12. The resilient medical device of claim 1 which is comprised of an outer resilient surface and an attachable or securing component to secure itself to the medical device.

13. The resilient medical device of claim 12, wherein the attachable or securing component is adhesive.

14. The resilient medical device of claim 12, wherein the attachable or securing component is a VELCRO substrate.

15. The resilient medical device of claim 1 which has a contour, shape, and size that is specific to the specific contour, shape, and size of the firmer hand-held medical device.

16. The resilient medical device of claim 1 which is two-dimensional in design and wraps around a medical device and secures one end to another via adhesive, Velcro, hook, fastener, or some other attachment to provide a cushioned feel over the handheld component of a firm medical device.

17. A resilient medical device which contains at least one control, button, switch, or sensor to add functionality to a firmer medical device that communicates with another component not directly located on the firmer medical device.

18. The resilient medical device of claim 17, wherein the other component is a switching mechanism or controller.

19. The resilient medical device of claim 17, wherein a gripping means suitable for the handle of an ablation catheter comprises an actuator that communicates with ablation therapy.

20. The resilient medical device of claim 19, wherein the actuator immediately disconnects ablation therapy.
